(19) United States
(12) Patent Application Publication
(10) Pub. No.: US 2026/0191763 A1
(43) Pub. Date: Jul. 9, 2026

(54) LIQUID COSMETIC FOR LIPS

(71) Applicant: SHISEIDO COMPANY, LTD., Chuo-ku, Tokyo (JP)

(72) Inventors: Noriko TOMITA, Tokyo (JP); Mizuki YAMAMOTO, Tokyo (JP)

(73) Assignee: SHISEIDO COMPANY, LTD., Chuo-ku, Tokyo (JP)

(21) Appl. No.: 19/133,026

(22) PCT Filed: Dec. 14, 2023
(86) PCT No.: PCT/JP2023/044906

§ 371 Date: May 27, 2025

(30) Foreign Application Priority Data
Dec. 28, 2022 (JP) 2022-212072

(51) Int. Cl. A61K 8/92; A61K 8/22; A61K 8/60; A61Q 1/04

(52) U.S. Cl. CPC A61K 8/922; A61K 8/22; A61K 8/60; A61Q 1/04; A61K 2800/43; A61K 2800/612; A61K 2800/651

(57) ABSTRACT

The present invention addresses the problem of providing a liquid cosmetic for lips having further improved transfer resistance (secondary adhesion-less effect). The present invention relates to a liquid cosmetic for lips, characterized by containing (A) at least one compound selected from (a1) copolymers containing dimeric acid esters and (a2) dimeric acid esters, (B) a non-volatile hydrocarbon oil that separates from (A) when being mixed with (A) at 25° C., (C) a gelling agent selected from dextrin fatty acid esters and waxes and (D) a color material. The color material (D) is preferably surface-treated with an amino acid or a metallic soap.

### TECHNICAL FIELD

The present invention relates to a liquid cosmetic for lips (a solid lip cosmetic). More specifically, it relates to a liquid cosmetic for lips with good usability, makeup durability, and color development, and in particular, exceptional transfer resistance (“secondary adhesion-less effect” or “secondary adhesion resistance effect”).

### BACKGROUND ART

When makeup cosmetics such as lipstick come into contact with dishes or clothing, the color may transfer (secondary adhesion) to the contact objects. Secondary adhesion not only makes the color of the makeup adhere on the contact object, but can also be a cause of messy makeup. Lipstick, in particular, is prone to secondary adhesion as the lips frequently come into contact with dishes. Therefore, cosmetics have been developed with so-called secondary adhesion-less effect, in which color transfer due to secondary adhesion is suppressed.

Patent Document 1 describes a liquid oil-in-oil cosmetic in which (a) a non-volatile hydrocarbon oil, (b) methylphenylpolysiloxane, and (c) a dextrin fatty acid ester are each blended in predetermined amounts, and the blending ratio (mass ratio) of (a) the non-volatile hydrocarbon oil to (b) methylphenylpolysiloxane is within a specific range.

Since (a) the non-volatile hydrocarbon oil and (b) methylphenylpolysiloxane are poorly compatible with each other, compositions containing both are prone to separate. However, in the cosmetic of Patent Document 1, a stable oil-in-oil composition is achieved by further blending (c) a dextrin fatty acid ester. On the other hand, when the cosmetic is applied to the lips, or the coated lips are pressed together, methylphenylpolysiloxane (referred to as “bleed oil” or “coating oil”) bleeds to the surface layer and separates, which covers the non-volatile hydrocarbon oil (referred to as “adhesion oil”), thus providing transfer resistance and good gloss. In addition, spreading during application can be improved while maintaining the transfer resistance and stability after application by blending a volatile hydrocarbon such as isododecane, which is compatible with both the above (a) and (b).

However, it was necessary to blend a large amount of silicone oil as coating oil, and this was not in line with silicone-free cosmetics, which have received attention in recent years.

The cosmetic for lips described in Patent Document 2 is solid but is said to have excellent glossy appearance in addition to secondary adhesion-less effect by using a copolymer containing a dimer acid ester and/or a dimer acid ester as an adhesion oil, and a hydrocarbon oil as a coating oil. However, liquid cosmetics formulated with such adhesion and coating oils have not been obtained.

In the solid cosmetic of Patent Document 2, problems such as stickiness and reduced glossy appearance caused by the waxes or the like blended to maintain solidity sometimes occurred. In addition, since the adhesion oil and the coating oil are in contact with each other, some of the color material will inevitably transfer from the adhesion oil to the coating oil. Therefore, it was difficult to completely prevent the color material that had transferred to the coating oil from being transferred to the contact object (secondary adhesion).

### CITATION LIST

### Patent Document

Patent Document 1: JP-B 4766720
Patent Document 2: JP-A 2022-115395

### SUMMARY OF INVENTION

### Technical Problem

In view of the current state of the arts described above, the present invention aims to provide a liquid cosmetic with good usability, makeup durability, and color development, as well as excellent transfer resistance (secondary adhesion-less effect).

### Solution to Problem

As a result of extensive studies to solve the aforementioned problem, the present inventors have found that the problems of usability and color development can be solved by replacing the wax in the solid cosmetic as described in Patent Document 2 with another gelling agent or by adjusting the content to prepare it as a liquid, and thereby completed the present invention.

Furthermore, the transfer of the color material from the adhesion oil to the coating oil can be suppressed and the transfer resistance (secondary adhesion-less effect) can be further enhanced by appropriately selecting a surface treatment of the color material to be blended in the cosmetic.

That is, the present invention provides a liquid cosmetic for lips, comprising:
(A) at least one selected from (a1) a copolymer comprising a dimer acid ester and (a2) a dimer acid ester;
(B) a non-volatile hydrocarbon oil that separates when mixed with (A) at 25° C.;
(C) a gelling agent selected from a dextrin fatty acid ester and a wax; and
(D) a color material.

### Advantageous Effects of Invention

The liquid cosmetic for lips of the present invention has excellent adhesion and glossy appearance by being formulated with a copolymer containing a dimer acid ester and/or a dimer acid ester as the adhesion oil. In addition, since a hydrocarbon oil is used as the coating oil, the content of silicone oil can be reduced. In addition to this, appropriately selecting the surface treatment of the color material allows to suppress the transfer of the color material to the coating oil, resulting in a cosmetic with exceptional makeup durability and secondary adhesion-less effect (transfer resistance).

### DESCRIPTION OF EMBODIMENTS

The liquid cosmetic for lips of the present invention (hereinafter, also simply referred to as “cosmetic”) contains, as essential components, (A) at least one selected from (a1) a copolymer containing a dimer acid ester and (a2) a dimer acid ester; (B) a non-volatile hydrocarbon oil that separates when mixed with (A) at 25° C.; (C) a gelling agent selected from a dextrin fatty acid ester and a wax; and (D) a color material.

The “component A” (also referred to as “adhesion oil”) of the cosmetic of the present invention is at least one selected from (a1) a copolymer containing a dimer acid ester and (a2) a dimer acid ester.

### (a1) Copolymer Containing Dimer Acid Ester

The “copolymer containing a dimer acid ester” (hereinafter, also referred to as “component a1”) is a high-viscosity, non-volatile ester oil. As the copolymer containing a dimer acid ester (component a1), it is preferable to select one that separates when heated to 90° C. with (B) the non-volatile hydrocarbon oil described below, mixed by stirring, then left to stand, and the mixture has reached 25° C.

Examples of the copolymer containing a dimer acid ester (component a1) include polyglyceryl-2 isostearate/dimer dilinoleate copolymer, with commercially available products including HAILUCENT ISDA (manufactured by Kokyu Alcohol Kogyo).

### (a2) Dimer Acid Ester

The dimer acid ester (hereinafter, also referred to as “component a2”) is an ester of a liquid fatty acid which has as a main component, a dibasic acid, C36 dicarboxylic acid, produced by dimerization of a C18 unsaturated fatty acid derived from a vegetable oil or fat, and contains monobasic and tribasic acids. Examples of the dimer acid ester (component a2) include hydrogenated castor oil dimer dilinoleate and dimer dilinoleyl dimer dilinoleate. Examples of commercially available products include RISOCAST DA-L (manufactured by Kokyu Alcohol Kogyo) and RISOCAST DA-H (manufactured by Kokyu Alcohol Kogyo).

The “component A” (adhesion oil) in the cosmetic of the present invention may contain either (a1) or (a2) alone, or may contain both (a1) and (a2).

In the present invention, it is preferable to contain (a1) a copolymer containing a dimer acid ester, and that the content of the (a1) copolymer containing a dimer acid ester be 50 mass % or more of the total amount of the “component A” (adhesion oil).

The content of the “component A” (total content of the component a1 and component a2) in the cosmetic of the present invention is 10 to 80 mass %, and from the viewpoint of color development, preferably 20 to 70 mass %, and even more preferably 25 to 60 mass % with respect to the total amount of the cosmetic.

### (B) Non-Volatile Hydrocarbon Oil

The “component B” (also referred to as “coating oil”) of the cosmetic of the present invention is a “non-volatile hydrocarbon oil that separates when mixed with the “component A” at 25° C.” (hereinafter, also simply referred to as “non-volatile hydrocarbon oil”), and is a non-volatile hydrocarbon oil which is in liquid form at normal temperature and can be used in a cosmetic.

The “component B” of the present invention separates when heated to 90° C. with the (A) copolymer containing a dimer acid ester and dimer acid ester, mixed by stirring, then left to stand, and the mixture has reached 25° C.

The terms “separate” and “compatible” in the present specification are defined as follows.
“Separate”: the “component A” and the “component B” were defined as “separating” if they separate into two layers with a uniform (visible) boundary when they were heated to 90° C. at (A):(B)=1:1 (mass ratio), mixed by stirring, then left to stand, and the mixture had reached 25° C., and they were defined as “not separating” if their mixture was transparent with no boundary.
“Compatible”: the “component A” and the “component B” were defined as “compatible” when they were mixed by stirring in the same manner at (A):(B)=1:1 (mass ratio), and the mixture at 25° C. was transparent with no visible boundary, i.e., “not separating.”

The non-volatile hydrocarbon oil (component B) used has a viscosity of 100 mPa·s or less, preferably 50 mPa·s. If the viscosity exceeds 100 mPa·s, a secondary adhesion-less effect may be less likely to be obtained. The viscosity in the present invention is the value measured with a B-type viscometer TVB-10M (manufactured by TOKI SANGYO CO., LTD.) at 25° C., rotor name M1, and 30 rpm.

Preferred specific examples of the non-volatile hydrocarbon oil (component B) include squalane, hydrogenated polyisobutene, hydrogenated polydecene, liquid paraffin, light isoparaffin, and heavy liquid paraffin. Examples of commercially available products include NOMCORT HP30 (manufactured by The Nisshin OilliO Group) and Squalane (manufactured by Kuraray).

The content of the “component B” (coating oil) in the cosmetic of the present invention is 10 to 80 mass %, preferably 20 to 70 mass %, and even more preferably 35 to 65 mass % with respect to the total amount of the cosmetic. If the content of the “component B” is less than 10 mass %, there will be less separation during application, and a secondary adhesion-less effect is less likely to be obtained. If the content exceeds 80 mass %, a secondary adhesion-less effect will be obtained, but sufficient color development is less likely to be obtained.

### (C) Gelling Agent

The gelling agent (also referred to as “component C”) blended in the cosmetic of the present invention is at least one selected from a dextrin fatty acid ester and a wax.

The dextrin fatty acid ester is an ester of dextrin and a higher fatty acid, where the higher fatty acid is selected from fatty acids with carbon numbers C12 to C22. The higher fatty acid may partly include fatty acids with carbon numbers C6 to C10 as long as it includes fatty acids with carbon numbers C12 to C22. Examples of such dextrin fatty acid ester include dextrin palmitate, dextrin myristate, and dextrin palmitate/ethylhexanoate, and commercially available products such as trade name Rheopearl KL, Rheopearl KL2, Rheopearl TT, Rheopearl TT2, and Rheopearl MKL2 (all manufactured by Chiba Flour Milling) can be used.

The wax used as (C) the gelling agent of the present invention is not particularly limited as long as it is commonly blended in a cosmetic. Among these, sunflower wax (sunflower seed wax), candelilla wax, microcrystalline wax, polyethylene wax, and sugar wax (such as sucrose tetrastearate triacetate) are preferably used.

The content of the gelling agent (component C) in the cosmetic of the present invention may be any amount that makes the cosmetic liquid. “Liquid” in the present invention means that the cosmetic is in a state having fluidity at normal temperature (25° C.) (liquid to paste-like). For example, “liquid” may be defined as having a viscosity in the range of 100 to 400,000 mPa·s.

For example, when a dextrin fatty acid ester is used as (C) the gelling agent, its content is 0.1 to 10 mass %, preferably 0.5 to 8 mass %, and more preferably 1 to 5 mass %. When a wax is used, its content is 0.1 to 6 mass %, and preferably 0.5 to 5 mass %. Too little gelling agent results in poor stability, and too much results in a sticky feeling.

### (D) Color Material

The color material (also referred to as “component D”) in the present invention is not particularly limited as long as it is a powdered color material. Examples thereof include inorganic white pigments (titanium dioxide and zinc oxide), inorganic red pigments (iron oxide (bengala) and iron titanate), inorganic brown pigments (gamma-ferric oxide), inorganic yellow pigments (yellow iron oxide and yellow ocher), inorganic black pigments (black iron oxide, carbon black, and lower titanium oxide), inorganic purple pigments (mango violet and cobalt violet), inorganic green pigments (chromium oxide, chromium hydroxide, and cobalt titanate), inorganic blue pigments (ultramarine and iron blue), pearl pigments (titanium oxide-coated mica, titanium oxide-coated bismuth oxychloride, titanium oxide-coated talc, colored titanium oxide-coated mica, bismuth oxychloride, and fish scale guanine), metal powder pigments (aluminum powder and copper powder), organic pigments (Red No. 202, Red No. 205, Red No. 220, Red No. 228, Red No. 405, Orange No. 203, Orange No. 204, Yellow No. 205, Yellow No. 40, and Blue No. 404), zirconium, barium and aluminum lake pigments (Red No. 3, Red No. 104, Red No. 227, Red No. 401, Orange No. 205, Yellow No. 4, Yellow No. 20, Green No. 3, and Blue No. 1), natural pigments (chlorophyll, carotenoids (β-carotene), carthamin, cochineal, chalcone, curcumin, betanin, flavonols, flavones, anthocyanidins, anthraquinone, and naphthoquinones), and functional pigments (boron nitride, photochromic pigments, synthetic fluorphlogopite, iron-containing synthetic fluorphlogopite, and hybrid fine powders).

The color material (component D) blended in the cosmetic of the present invention preferably includes color materials that have been surface-treated with a treatment agent containing an amino acid or a metallic soap. Blending a color material that has been surface-treated with a treatment agent containing an amino acid or a metallic soap allows to suppress the transfer of the color material from the adhesion oil to the coating oil, which further improves the secondary adhesion-less effect.

A treatment agent containing an amino acid is a surface-treating agent containing at least one amino acid or derivative thereof.

The “amino acid” is not particularly limited, but is preferably selected from aspartic acid, glutamic acid, lysine, proline, and sarcosine, of which glutamic acid is particularly preferred.

The “derivative of an amino acid” is preferably an N-acyl amino acid or a salt thereof. The “N-acyl amino acid or salt thereof” is a compound in which an acyl group, preferably a saturated fatty acid having 12 to 20 carbon atoms, is condensed on an amino group of an amino acid, or a salt thereof. Examples of the “acyl group” include a stearoyl group, a lauroyl group, a palmitoyl group, and a cocoyl group. The “salt” can be selected from alkali metal salts such as sodium and potassium, and alkaline earth metal salts, but is preferably a sodium salt. Specific examples of the N-acyl amino acid salt include disodium N-stearoyl glutamate, sodium N-lauroyl glutamate, sodium lauroyl aspartate, sodium palmitoyl sarcosinate, and magnesium palmitoyl glutamate, but are not limited thereto.

The treatment agent containing an amino acid may contain at least one other treatment agent in addition to the “amino acid or derivative thereof”. The “other treatment agent” is preferably selected from “metal hydroxides,” “lipids,” and “esters.” Examples of the “metal hydroxides” include aluminum hydroxide. Examples of the “lipids” include higher fatty acids such as “palmitic acid.” Examples of the “esters” include esters of monovalent or divalent fatty acids having 8 to 12 carbon atoms and saturated aliphatic alcohols having 12 to 20 carbon atoms. The alkyl chain of the fatty acids and aliphatic alcohols may be linear or branched. As the “ester,” isostearyl sebacate is particularly preferably used.

Examples of the “treatment agent containing an amino acid” include a surface-treating agent containing disodium N-stearoyl glutamate and aluminum hydroxide, a surface-treating agent containing disodium N-stearoyl glutamate, isostearyl sebacate, and aluminum hydroxide, a surface-treating agent containing sodium N-lauroyl glutamate and lysine, and a surface-treating agent containing palmitoyl proline, sodium palmitoyl sarcosinate, magnesium palmitoyl glutamate, and palmitic acid. In the present invention, it is particularly preferable to use a color material that has been surface-treated with a treatment agent containing disodium N-stearoyl glutamate or sodium N-lauroyl glutamate lysine.

The treatment agent containing a metallic soap is a surface-treating agent containing at least one metallic soap.

The “metallic soap” is a salt of a saturated or unsaturated higher fatty acid with a metal (but other than alkali metals). Although not particularly limited, examples of the higher fatty acids constituting the metallic soap include saturated and/or unsaturated higher fatty acids having 8 to 24, particularly 12 to 18 carbon atoms, such as stearic acid, isostearic acid, myristic acid, and lauric acid. A salt of aluminum, calcium, magnesium, or zinc is preferred as the metal constituting the metallic soap.

Specific examples of the metallic soap preferably used as a surface-treating agent of the color material (component D) in the present invention include magnesium stearate, calcium stearate, zinc stearate, calcium laurate, zinc laurate, calcium ricinoleate, zinc ricinoleate, zinc octylate, and aluminum myristate.

The surface treatment with a metallic soap can be performed by a method of surface-treating with a preformed metallic soap. For example, the surface treatment can be performed by dissolving a metallic soap in a volatile solvent such as isoparaffin or isopropyl alcohol, mixing it with a base powder (color material), and then volatilizing the volatile solvent. The surface treatment can also be performed by simply mixing a base powder (color material) with a metallic soap. Alternatively, the surface treatment may be performed by a method of composite treatment with a higher fatty acid (e.g., stearic acid) constituting a metallic soap, and a hydroxide of a metal (e.g., aluminum). The method of surface treatment used can be a wet method using a solvent, a gas phase method, a mechanochemical method, or the like, and is not particularly limited.

The color material (component D) of the cosmetic of the present invention may consist of one or more color materials surface-treated with a treatment agent containing an amino acid, or one or more color materials surface-treated with a treatment agent containing a metallic soap. Alternatively, a color material surface-treated with a treatment agent containing an amino acid may be blended in combination with a color material surface-treated with a treatment agent containing a metallic soap. In addition, the color material (component D) may further include a color material surface-treated with a treatment agent free of amino acids and metallic soaps and an untreated color material, in addition to a color material surface-treated with a treatment agent containing an amino acid and/or a metallic soap.

The content of the color material (component D) in the cosmetic of the present invention is preferably 0.1 to 15 mass %, more preferably 1 to 12 mass %, and even more preferably 4 to 10 mass % with respect to the total amount of the cosmetic. The color material surface-treated with a treatment agent containing an amino acid or a metallic soap preferably accounts for 50 mass % (mass ratio) or more, more preferably 60 mass % or more, and even more preferably 70 mass % or more of the total amount of the color material (component D) blended in the cosmetic.

In addition to the above components A to D, other optional components commonly used in liquid cosmetics can be blended into the liquid cosmetic for lips within a range not impairing the effects of the present invention. Examples of the other optional components include, but are not limited to, oils (other than the components A to C), powders (other than the component D), polymer compounds, moisturizers, fragrances, antioxidants, preservatives, and beauty ingredients.

Examples of powders (other than the component D) include silicic anhydride (silica).

The silicic anhydride is preferably ultrafine silicic anhydride with an average primary particle size of 1 to 50 nm. Examples of commercially available products include AEROSIL 200, 300, R972, R974 and RY200 (manufactured by Nippon Aerosil). The silicic anhydride used in the present invention may be hydrophilic or hydrophobically treated, for example, by silylation.

When silicic anhydride is blended, the preferred content is 10 mass % or less, preferably 8 mass % or less, and can be, for example, 0.1 to 5 mass %.

Powders other than the above include spherical powders and plate-like powders.

Examples of spherical powders include spherical resin powders such as polymethyl methacrylate, organopolysiloxane elastomer, polystyrene, polyamide resin (nylon), polyethylene, copolymer of styrene and acrylic acid, benzoguanamine resin, polytetrafluoroethylene, and silicone resin.

Examples of plate powders include inorganic powders such as mica, synthetic mica, talc, sericite, aluminum oxide, magnesium oxide, zirconium oxide, magnesium carbonate, calcium carbonate, calcium sulfate, chromium oxide, chromium hydroxide, aluminum silicate, magnesium silicate, magnesium aluminum silicate, kaolin, silicon carbide, barium sulfate, bentonite, smectite, and boron nitride, organic powders such as N-acyllysine, and hybrid powders such as fine titanium oxide-coated titanated mica, fine zinc oxide-coated titanated mica, and barium sulfate-coated titanated mica.

The content of the powders (other than the component D) in the cosmetic of the present invention is preferably 30 mass % or less, and even more preferably 20 mass % or less with respect to the total amount of the cosmetic.

The cosmetic of the present invention is produced using a method widely used for oil-based cosmetics. For example, the cosmetic can be produced by heating each formulation component as necessary, mixing by stirring, filling into a container, and cooling slowly.

The cosmetic of the present invention is an oil-in-oil emulsion liquid cosmetic using a dimer acid ester as an adhesion oil, and a non-volatile hydrocarbon oil as a coating oil.

The cosmetic of the present invention is suitable to be provided as a makeup cosmetic, particularly a liquid cosmetic for lips with exceptional transfer resistance (secondary adhesion-less effect). Specifically, it is preferably provided in the form of a liquid lipstick, lip gloss, lip base for priming, lipstick top coat, and lip balm.

### EXAMPLES

Hereinafter, the present invention will be described in further detail with reference to Examples, but the present invention is not limited to these Examples. Unless otherwise specified, the content is indicated in mass %.

Liquid cosmetics for lips were prepared according to a conventional method, using the formulations listed in Tables 1 and 2 below. The characteristics of the cosmetic of each example in Tables 1 and 2 were measured and evaluated by the following methods.

### <Test Method and Evaluation Criteria>

A real-world use test was conducted by a panel of 10 experts. The points of evaluation were secondary adhesion-less effect, lack of stickiness over time, durability of the finish, color development, and glossy appearance/moisture feeling after application, and each point of evaluation was given a 5-level sensory evaluation (score) based on the following evaluation criteria. The average of the scores was used to make an assessment according to the following evaluation criteria. The evaluations of the lack of stickiness over time and durability of the finish were the results of comparing the evaluation at the time of application or immediately after application with the evaluation several hours after application.

The method used for applying the cosmetic was a method in which the cosmetic of the present invention was applied to the lips and then pressure was applied for about 5 seconds by pressing the top and bottom of the lips together. Transfer resistance was evaluated based on the absence of transfer to the cup, and the other points were visually assessed by an expert panel.

### (Score)

5 points: Exceptionally excellent.
4 points: Excellent.
3 points: Average.
2 points: Poor.
1 point: Extremely poor.

### (Evaluation Criteria)

A: Evaluation value (average) was 4.0 points or higher
B: Evaluation value (average) was higher than 3.0 points and lower than 4.0 points
C: Evaluation value (average) was higher than 2.0 points and lower than 3.0 points
D: Evaluation value (average) was lower than 2.0 points

TABLE 1

| | Example | Example | Example | Example | Example | Example |
|---|---|---|---|---|---|---|
| | 1 | 2 | 3 | 4 | 5 | 6 |
| Polyglyceryl-2 isostearate/dimer | 10 | 30 | 60 | 80 | 30 | 30 |
| dilinoleate copolymer | | | | | | |
| Squalane | 80 | 60 | 30 | 10 | 60 | 60 |
| Dextrin fatty acid ester | 5 | 5 | 5 | 5 | 5 | 5 |
| Amino acid-treated iron oxide | 5 | 5 | 5 | 5 | — | — |
| (*1) | | | | | | |
| Metallic soap-treated iron oxide | — | — | — | — | 5 | — |
| (*2) | | | | | | |
| Silicone-treated iron oxide | — | — | — | — | — | 5 |
| Total | 100 | 100 | 100 | 100 | 100 | 100 |
| Secondary adhesion-less | A | A | A | A | B | B |
| effect | | | | | | |
| Lack of stickiness over time | A | A | A | A | A | A |
| Long-lasting effect of finish | A | A | A | A | A | B |
| Color development | B | A | A | A | A | B |
| Glossy appearance/moisture | A | A | A | A | A | A |
| feeling after application | | | | | | |

(*1) Iron oxide surface-treated with disodium stearoyl glutamate
(*2) Iron oxide surface-treated with magnesium stearate

TABLE 2

| | Example | Example | Example | Example | Example |
|---|---|---|---|---|---|
| | 7 | 8 | 9 | 10 | 11 |
| Polyglyceryl-2 isostearate/dimer | — | 30 | 30 | 30 | 30 |
| dilinoleate copolymer | | | | | |
| Hydrogenated castor oil dimer | 30 | — | — | — | — |
| dilinoleate | | | | | |
| Squalane | 60 | 61 | 61 | 61 | 61 |
| Dextrin fatty acid ester | 5 | — | — | — | — |
| Sunflower seed wax | — | 4 | — | — | — |
| Candelilla wax | — | — | 4 | — | — |
| Polyethylene/microcrystalline wax | — | — | — | 4 | — |
| Sucrose tetrastearate triacetate | — | — | — | — | 4 |
| Amino acid-treated iron oxide (*1) | 5 | 5 | 5 | 5 | 5 |
| Total | 100 | 100 | 100 | 100 | 100 |
| Secondary adhesion-less effect | A | A | A | A | A |
| Lack of stickiness over time | A | A | A | A | A |
| Long-lasting effect of finish | A | A | A | A | A |
| Color development | A | A | A | A | A |
| Glossy appearance/moisture | A | A | A | A | A |
| feeling after application | | | | | |

(*1) Iron oxide surface-treated with disodium stearoyl glutamate

The test results shown in Tables 1 and 2 confirm that the oil-in-oil emulsion liquid cosmetic of the present invention is sufficiently superior in all the evaluated points. An equally excellent effect was obtained even when varying the content ratio of the adhesion oil (component A) to the coating oil (component B) over a wide range of 1:8 to 8:1.

On the other hand, the examples using a color material treated with an amino acid were superior on all points, including the secondary adhesion-less effect and color development, but with a color material treated with a metallic soap, the secondary adhesion-less effect was slightly reduced. When a color material treated with silicone was used, the secondary adhesion-less effect, makeup durability (long-lasting effect), and color development were slightly reduced. Therefore, selecting an amino acid-treated or metallic soap-treated color material in the cosmetic of the present invention allowed to obtain a cosmetic with particularly excellent color development, and in particular, selecting an amino acid-treated color material allowed to obtain a cosmetic which achieves both excellent secondary adhesion-less effect and color development.

Although data are not shown, replacing a portion of the squalane in Example 10 with a wax and adjusting the amount of wax so that the viscosity of the cosmetic at 25 C exceeded 400,000 mPa·s resulted in a sticky feeling, and the cosmetic was inferior in color development, glossy appearance, and moisture feeling.

1. A liquid cosmetic for lips, comprising:
(A) at least one selected from (a1) a copolymer comprising a dimer acid ester and (a2) a dimer acid ester;
(B) a non-volatile hydrocarbon oil that separates when mixed with (A) at 25° C.;
(C) a gelling agent selected from a dextrin fatty acid ester and a wax; and
(D) a color material.

2. The cosmetic according to claim 1, wherein the (D) color material is a color material surface-treated with a treatment agent comprising an amino acid or a metallic soap.

3. The cosmetic according to claim 2, wherein the (D) color material is a color material surface-treated with a treatment agent comprising an N-acyl amino acid or a salt thereof.

4. The cosmetic according to claim 3, wherein the N-acyl amino acid is N-stearoyl glutamic acid.

5. The cosmetic according to claim 2, wherein the (D) color material is a color material surface-treated with a treatment agent comprising a metallic soap.

6. The cosmetic according to claim 5, wherein the metallic soap is at least one selected from the group consisting of magnesium stearate, calcium stearate, zinc stearate, calcium laurate, zinc laurate, calcium ricinoleate, zinc ricinoleate, zinc octylate, and aluminum myristate.

7. The cosmetic according to claim 1, wherein the (a1) copolymer comprising a dimer acid ester is a polyglyceryl-2 isostearate/dimer dilinoleate copolymer.

8. The cosmetic according to claim 1, wherein the (a2) dimer acid ester is selected from hydrogenated castor oil dimer dilinoleate and dimer dilinoleyl dimer dilinoleate.

9. The cosmetic according to claim 1, wherein the (B) non-volatile hydrocarbon oil is selected from squalane, hydrogenated polyisobutene, hydrogenated polydecene, liquid paraffin, light isoparaffin, and heavy liquid paraffin.

10. The cosmetic according to claim 1, wherein the (C) gelling agent is at least one selected from a dextrin fatty acid ester, sunflower wax, candelilla wax, microcrystalline wax, polyethylene wax, and sugar wax.

11. The cosmetic according to claim 1, wherein a viscosity is in the range of 100 to 400,000 mPa·s.
